(19) United States
(12) Patent Application Publication
(10) Pub. No.: US 2002/0168403 A1
(43) Pub. Date: Nov. 14, 2002

(54) Compositions and therapy for substance addiction

(76) Inventor: Roni Biberman, Tel-Aviv (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 10/086,978

(22) Filed: Feb. 28, 2002

Related U.S. Application Data
(63) Continuation-in-part of application No. 09/898,027, filed on Jul. 5, 2001.
(60) Provisional application No. 60/216,366, filed on Jul. 5, 2000.

(51) Int. Cl. A61K 31/137; A61K 9/48; A61K 9/20

(52) U.S. Cl. 424/452; 424/465; 514/649

(57) ABSTRACT

Inhibitors of monoamine oxidase used in combination with an addictive substance, or a pharmacological derivative or analogue thereof, are useful for the treatment of substance addiction disorders. In particular, the invention discloses compositions, and methods of use thereof, comprising selegiline and nicotine for the treatment of cigarette smokers wishing to abstain. The compositions and methods of use thereof include oral, inhalant, parenteral and transdermal patch modes of therapy, whereby the subject benefits from the combined effects of a monoamine oxidase inhibitor in combination with an addicitive substance, or derivative thereof.

This Application is a continuation-in-part Application of U.S. patent application Ser. No. 09/898,027, filed on Jul. 5, 2001 and which is hereby incorporated herein by reference. This Application claims priority from U.S. Provisional Patent Application No. 60/216,366, filed on Jul. 5, 2000 which is hereby incorporated herein by reference.

### FIELD OF THE INVENTION

The present invention relates to medicinal compositions and methods of treating substance addiction, in particular addiction to nicotine-containing tobacco products. The present invention is related to co- administration of inhibitors of monoamine oxidase with an addictive substance in order to alleviate withdrawal symptoms and to change or eliminate patterns of addictive substance consumption.

### BACKGROUND OF THE INVENTION

Both nicotine and tobacco smoke are considered to be dangerous to health. Nicotine has vasoconstrictive properties and probably accounts for the substantial increased risk for smokers to develop ischemic vascular disease, leading to heart disease, stroke, and peripheral vascular disease. Tobacco smoke contains a multitude of known carcinogens, and as well, raises blood carbon monoxide levels which impact negatively on hemoglobin function. Acute and chronic opiate, amphetamine, cocaine and ethanol use are also known to produce cardiovascular disorders.

The statistical risk of dying from lung cancer in the United States has doubled in the past thirty years for male cigarette smokers and has quadrupled for female cigarette smokers. Lung cancer has now displaced cardiovascular disease as the single most important cause of excess mortality among smokers. Yet, about 50 million Americans continue to smoke.

The benefits for smoking cessation are many, and are summarized in a publication entitled “The Health Benefits of Smoking Cessation: A Report of the Surgeon General, 1990”, available from the Office on Smoking and Health, Center for Disease Control, Rockville, Md. Among the benefits summarized are that within twenty-four hours the chance of a heart attack decreases, within about two weeks to three months lung function increases up to thirty percent, and in one year the excess risk of coronary heart disease becomes half that of a smoker.

Smoking cessation programs often address both the physiological factor and the psychological factor. The widely available “nicotine-releasing patches” have been highly advertised, and are useful for maintaining a nicotine-habituated patient on nicotine while addressing further the psychological factors of addiction. Craving is the most consistently observed withdrawal symptom and is probably best described as a combination of physiological and psychological factors. To treat craving for nicotine, other modes of nicotine replacement therapy have been introduced, including adminstration of nicotine by oral chewing gum and by inhalation with a carrier for absorption through the respiratory mucosa, including the lungs.

In addition to nicotine replacement therapy, other pharmacological therapies have been described based on compounds which are expected to influence the physiological mechanisms common to nicotinic agents. This is the basis for the use of compounds which interact with acetylcholine receptors, or affect levels of the neurotransmitter acetylcholine, as a means for reducing the desire for nicotine or other addictive drugs. Atropine and scopolamine are alkaloids that block the action of acetylcholine at muscarinic receptors to produce antispasmodic, antisecretory, and antiparkinsonism actions. Physostigmine is an inhibitor of the enzyme acetylcholinesterase, thereby raising the level of acetylcholine through inhibition of its metabolism.

Other compounds in addition to cholinergic and anticholinergic drugs have been tested for the purpose of attenuating withdrawal symptoms, including craving. These include benzodiazepines, dopamine agonists, serotonin agonists, inhibitors of dopamine carboxylase, and inhibitors of serotonin reuptake or of dopamine reuptake. The neurotransmitter dopamine has been shown to be strongly linked to the internal mechanism of pleasure sensation mechanism of animal, including human brains.

Inhibitors of monoamine oxidase (MAO), are generally employed in the medical arts as anti-depressants and as anti-parkinson agents, but the use in combination with administration of an addictive substance, or derivative thereof, for treatment of addiction is novel. The use of MAO inhibitors in the treatment of addiction, in particular, tobacco product addiction, has been recurrently suggested although, for lack of conclusive data, these therapeutic compounds are not currently endorsed for this use by the medical establishment.

The goal of therapy or treatment of substance addiction is to reduce the amount and/or rate of intake of the substance over time and as well, to reduce the rate of relapse. It is well known that individuals afflicted with an addictive condition who succeed in obtaining a reduction or complete cessation of intake of the substance to which they are addicted remain at a substantial risk to relapse during the course of their lifetime. To cure, or completely eradicate, an addictive condition over the course of the lifetime of a subject afflicted with a substance addiction often predicates life-long administration of therapy, be it pharmacological, behavioral, or both.

One important measure of the efficacy of a therapy for addiction is the level of desire for ingesting the substance, otherwise known as craving. Most addictive substances are clearly associated with the condition of craving following a brief period of abstinence, ranging from minutes to days, depending upon the substance and the individual. For many afflicted individuals, the phenomenon of craving may be lifelong, thus leading to the need for prolonged administration of therapy for addiction.

While craving is considered to be a psychological or strong mental desire which is difficult to resist, other aspects of substance addiction which should be targeted by specific therapies are other acute and chronic withdrawal symptoms in addition to craving. In the case of cocaine addiction, the most prominent withdrawal symptoms are subjective sensations of mild to severe dysphoria, depression, anxiety or irritability. This is also true of amphetamine, alcohol, opiate and nicotine withdrawal. Thus, in addition to reducing intake or craving, another measure of the efficacy of anti-addiction therapy is to diminish the undesirable psychological effects associated with discontinuation or withdrawal from the addictive substance or product.

### SUMMARY OF THE INVENTION

In one embodiment, this invention provides a composition for the treatment of addiction to an addictive substance comprising an effective amount of an inhibitor of monoamine oxidase, an effective amount of an addictive substance, or derivative thereof, and a suitable diluent or carrier. The inhibitor of monoamine oxidase and the addictive substance may be administered via a single vehicle or co-administered via different vehicles. Furthermore, the inhibitor of monoamine oxidase and the addictive substance may be administered via the same route of administration or via different routes of administration.

In one embodiment, this invention provides a pharmaceutical composition comprising an effective amount of an inhibitor of monoamine oxidase, an effective amount of an addictive substance or derivative thereof, and a suitable diluent or carrier, wherein the vehicle of said composition is an oral tablet, a capsule, a comestible article, an aerosol, a nebulized vapor, a smoke, or a transdermal patch, and wherein said vehicle is suitable for parenteral or oral administration. Parenteral administration includes intradermal, subcutaneous, intramuscular, intravenous, intrathecal, sublingual, rectal, vaginal, intraocular, transdermal, respiratory mucosal, or pulmonary routes of administration.

In one embodiment, this invention further provides a method of treating a subject addicted to an addictive substance or product comprising the step of co-administering to the subject in need thereof a pharmaceutical composition comprising a therapeutically effective amount of monoamine oxidase inhibitor and a therapeutically effective amount of an addictive substance, thereby attenuating consumption of said substance or product. In a preferred embodiment, said monoamine oxidase inhibitor is selegeline and said additive substance is nicotine. As used herein, the term “attenuating consumption” referrs to decreasing consumption of said substance by at least about 10%, preferably at least about 30%, at least about 50%, at least 70%, and up to 100%.

In one embodiment, this invention provides a method of treating the symptoms of nicotine withdrawal such as chronic craving symptoms, comprising the step of co-administering to the subject in need thereof a pharmaceutical composition comprising a therapeutically effective amount of monoamine oxidase inhibitor and a therapeutically effective amount of nicotine, thereby alleviating the symptoms of nicotine withdrawal. In a preferred embodiment, said monoamine oxidase inhibitor is selegeline.

In one embodiment, this invention further provides a method of treating a subject addicted to a tobacco product comprising the step of co-administering to the subject in need thereof a pharmaceutical composition comprising a therapeutically effective amount of monoamine oxidase inhibitor, preferably selegiline, and a therapeutically effective amount of nicotine, thereby attenuating tobacco consumption.

### DETAILED DESCRIPTION OF THE INVENTION

In one embodiment of the invention, a method for relieving craving in a nicotine-habituated person who is abstaining from or reducing nicotine intake is provided by administering at least two components individually or as an admixture of first and second components The first component is an inhibitor of monoamine oxidase and the second component is an addictive substance or a non-addicitive derivative thereof. Co-administration of the two components or administration of a balanced admixture of the first and second components relieves craving, yet surprisingly results in substantially no undesirable physiological changes. Instead, practice of the invention appears to reduce not only craving for nicotine, or nicotine-containing tobacco products, in persons who are reducing or abstaining from nicotine intake, but appears as well to reduce other cravings associated with withdrawal syndromes, such as weight gain. The invention is thus useful for treating a wide range of withdrawal syndromes, including those encountered in subjects addicted to opiates, amphetamines, or cocaine.

In one embodiment, this invention provides the administration of the monoamine oxidase inhibitor selegiline, which is specific in low doses for the monoamine oxidase-B isoform of the enzyme, is comparable to nicotine replacement therapy for the treatment of tobacco product addiction. Of paramount significance is the finding, as disclosed herein, that when administered as combination therapy, selegiline and nicotine are synergistic in the ability to treat the affliction of tobacco product addiction, the combination achieving greater reductions in cigarette consumption than would be expected from the sum of each form of therapy administered individually.

Combination therapy comprised of administration of both nicotine and selegiline together, by either identical or separate routes of administration, is disclosed herein as a highly effective method for the treatment of tobacco addiction, exhibiting a synergistic effect in comparison to that of administration of either agent alone and achieving Continous Abstinance of greater than 30%.

A particularly preferred composition of the invention takes the form of a tablet, a capsule, or a comestible artcle for oral administration or of a patch for transdermal administration wherein the first component is an inhibitor of monoamine oxidase and the second component is an addictive substance, such as nicotine, or pharmacological analogue thereof. The combination of the first and second components is useful and effective because the first class of drugs (specific or non-specific inhibitors of monoamine oxidase) should influence the response to nicotine, or a similarly addictive substance, through affects on neurotransmitter pathways or metabolism. As demonstrated herein, the inhibitor of monoamine oxidase selegiline is effective in itself to reduce the desire or craving for an addictive substance, namely nicotine. Furthermore, administration of the inventive compositions comprising both classes of compounds, or alternatively, co-administration of both classes of compounds by different vehicles, have been shown to lead to a reduced desire for nicotine, which is greater than when subjects are administered a comparative mode of therapy using only nicotine or selegeline alone.

The invention disclosed herein comprises the combination of a monoamine oxidase inhibitor, such as selegiline, with one or more modes of nicotine replacement therapy, for the treatment of addiction, in particular addiction to tobacco products. In particular the invention provides a pharmaceutical composition comprising an addictive substance, or a pharmaceutical analogue or derivative thereof, in combination with an inhibitor of monoamine oxidase, for administration to the same subject as a pharmacological treatment, employed alone or as an adjunctive measure, to promote the process of withdrawal from habitual consumption of an addictive substance or product wherein an addictive substance is contained.

The terms addiction, substance addiction, substance abuse, or substance dependence are frequently used interchangeably. In general, the term substance addiction is used herein to refer to the chronic administration, usually self-administration, of a chemical compound, or class of chemical compounds, which is either itself known to exert noxious effects to health and/or the route of administration of the compound or product thereof creates substantial physiological insults, yet the subject afflicted with the addiction continues to crave, seek, or administer the substance despite the known risks to health. Such is the case with tobacco product addiction whereby the active substance, nicotine, is most typically administered via the act of inhalation of tobacco smoke, as in cigarette, cigar, or pipe smoking. Nicotine is known to be highly addictive, hence the efforts to aid the afflicted subject via administration of purified, concentrated nicotine in the form of a transdermal patch, buccoral, oral, or respiratory route which obviate the need to inhale noxious tobacco smoke for acquiring the desired dose of nicotine. Thus, a subject addicted to cigarette smoking, or another tobacco product, may make use of the invention while attenuating or completely ceasing consumption of the offending tobacco product, and yet avoid the withdrawal signs and symptoms associated with this action, especially craving, thus allowing the addicted subject to avoid complete or partial relapse, whereby the addicted subject reinstitutes the addictive behaviors associated with the addictive substance, such as cigarette smoking.

The term substance dependence usually is taken to refer to the psychological and behavioral aspects of chronic ingestion of a substance known to have adverse effects on health or daily functioning. Substance dependence is thus one specific aspect of substance addiction.

The term substance abuse usually is taken to refer to periodic or chronic consumption of a substance, in particular a psychoactive substance, the consumption of which is continued by the subject despite the presence or knowledge of the associated harmful effects. Substance abuse is thus one specific aspect of substance addiction.

An addictive substance is any compound, combination of compounds, or product thereof, known to be addictive to an animal subject, especially human beings. An addictive substance is often consumed by the addicted subject in a repetitive, compulsive manner in order to obtain a mood-enhancing or mood-stimulating effect or to diminish or postpone the mood-related dysphoria and other psychological, emotional, and physical effects experienced by the addicted subject upon discontinuation or withdrawal from the ordinary schedule of consumption of the addictive substance. Craving, defined as the desire for the addictive substance and/or the desire to partake of the behavior patterns associated with consumption of the addictive substance, is a critical feature of addictive disorders. Examples of addictive substances are opiates, including heroin, morphine, codeine, and natural or synthetic derivatives thereof; opiate-like analgesic drugs, including fentanyl, buprenorphine, and natural or synthetic derivatives thereof; nicotine and natural or synthetic derivatives thereof; tobacco and tobacco-containing products thereof; cocaine and natural or synthetic derivatives thereof; amphetamines and natural or synthetic derivatives thereof; ethyl alcohol and ethanol-containing products; caffeine and caffeine-containing products; and high carbohydrate diets, especially the habitual consumption of sugar products and sweets.

For implementation of the compositions and methods of use thereof provided by the invention, a non-addictive derivative or analogue may be used in place of, or in addition to, the addictive substance. As defined herein, a “derivative thereof” of an addictive substance is a substance which shares relevant pharmacological properties in common with a particular addictive substance, so that the “derivative thereof” may be considered to be either a pharmacological analogue, partial analogue, or antagonist of the addictive substance, but which is either less addictive or non-addictive in comparison to said addictive substance when tested in animals, including humans. Thus, for example, methadone or naloxone may be used in addition to or instead of morphine, heroin, or another highly addictive opiate.

Addictions are associated with specific symptoms of withdrawal, depending upon the substance for which addiction had developed. For example, cigarette smokers who reduce or cease tobacco product consumption frequently experience increased appetite for food and therefore are at risk for weight gain. In fact many smokers report that the experience or fear of weight gain significantly contributes to their inability to reduce or cease their level of tobacco product consumption. Cocaine, amphetamine, and opiate habits are also initiated and promulgated for their weight loss potential. Thus the invention provides composition and therapeutic methods for the control of body weight in a subject addicted to a chemical substance or product thereof, including nicotine, amphetamines, cocaine, and opiates.

In addition, the well-known phenomena of compulsive consumption of sweets, although not formally classified as a substance addiction disorder by all medical disciplines, has many aspects in common with other addictive disorders. Thus, subjects afflicted with an addiction to frequent episodes of craving for food, in particular sweets, may benefit from the anti-craving effects of the invention.

Other well-known withdrawal symptoms associated with substance addiction are headache, sleep disturbances, including hypersomnia and insomnia, and signs of autonomic arousal such as sweating, tachycardia, diarrhea, stomach cramps, nausea and vomiting.

Nicotine addiction, as exemplified by chronic cigarette smoking, may be taken to be a valid model for other conditions of substance addiction. Many substance addictions share certain aspects in common, as delineated above with respect to craving and weight gain as withdrawal phenomena. Thus the compositions and methods for treatment of addiction disclosed herein may be considered useful for the treatment of substance addiction in general, particularly since they are believed to share a common physiological mechanism attributed to the dopaminergic, and other neurotransmitter, pathways of reward in the brain.

Inhibitors of the intracellular enzyme monoamine oxidase have been suggested to be of value in the treatment of addiction, although to date this contention has never been rigorously established by way of collection and analysis of clinical data. An inhibitor of monoamine oxidase is a compound which either inhibits in vitro the reaction catalyzed by the enzyme, or a compound which binds to the enzyme as demonstrated by in vitro or in vivo binding analysis, For purposes of the present invention, a monoamine oxidase inhibitor may be either a reversible or an irreversible inhibitor of the enzyme. Similarly, a monoamine oxidase inhibitor may be either a selective or a non-selective inhibitor with respect to the two known isoforms of the enzyme, monoamine oxidase A and monoamine oxidase B. Examples of monoamine oxidase inhibitors include, but not only, the following: selegiline (deprenyl, I-deprenyl) (R)-(−)-N,2-dimethyl-N-2-propynylphenethylamine; desmethylselegiline; S(+)desmethylselegiline, lazabemide; broformine; rasagiline; LU-53439; LU43839; idasoxan; clorgyline (N-[3-(2′,4′-dichloro-phenoxy)-N-methylpropargylamine) (May & Baker); cimoxatone, cirazoline (MD780515, MD-770222) (Synthelabo); befloxatone (MD-370503) (Synthelabo); brofaromine (Consonar, CGP-11305A) (Ciba-Geigy); bazinaprine (SR 95191) (3-(2-morpholino-ethylamino)4-cyano6-phenyl-pyridazine) (Sanofi); MD-240928 (R-3-[4-((3-chlorophehyl)methoxy)phenyl]-5-[(methylamino)methyl]-2-oxazolidinone methanesulfonate (Lilly); BW-616U (Burroughs Wellcome); BW-1370U87 (1-ethylphenoxathiin-10,10-dioxide (Burroughs Wellcome); CS-722 (RS-722) ((R)-4-chloro-2-(2-hydroxy-3-morpholinopropyl)-5-phenyl-4-isoxazolin-3-one hydrochloride (Sankyo); E-2011 ((5R)-3-[2-((1S)-3-cyano-1-hydroxypropyl)-benzothiazol-6-yl]-5-methoxymethyl-2-oxazolindinone (Eisai); harmine (3-phenylquinoline,3-(4-hydroxyphenyl)quinoline; harmane (1-methyl-9H-pyridol/3,4-b/indole); norharmane; harmaline; moclobemide (Aurorix, RO 11-1163) (Roche); minaprine (3-(2-morpholinoethylamino)4-methyl-6-phenyl- pyridazine; PharmaProjects 3975 (Hoechst); RO 41-1049 (N-(2-aminoethyl)-5-(3-flurophenyl)-4-thiazolecarboxemide) (Roche); RS-8359 (+/−)4-(4-cyanoanilino)-5,6-dihydro-7-hydroxy-7H-cyclopenta[d]-pyrimidine) (Sankyo); T-794 ([(5R)-3-(6-(cyclcopropylmethoxy)2-napthealenyl)-5-(methoxymethyl)2-oxazolidone) (Tanabe Seiyaku); toloxatone (Humoryl, Perenum, MD-690276, MD-69276) (Synthelabo); K-Y 1349 (Kalir and Youdim); LY-51641 (N-[2-(o-chlorophenylxoy)ethyl]cyclopropylamine hydrochloride) (Lilly); LY-121768 (N-[2-o-iodophenoxy)ethyl]cyclopropylamine hydrochloride) (Lilly); M&B 9303 (May & Baker); MDL 72394 (Marion Merrell); MDL 72392 (Marion Merrell); sercloremine (CGP4718A) (Ciba-Geigy); MD 72394 ((E)-betafluoroethylene-m-tyrosine); MO 1671; amiflamine ([S-(+)4-dimethylamino-alpha,2-dimethylphenetrhylamine] (FLA-336, FLA-668, FLA-788) (Astra); vanoxerine (boxeprazine) (Novo-Nordisk); AGN 2253 (Nicholas Kiwi); iproniazid (Marsilid); isocarboxazid (Marplan) (Roche); M-3-PPC (Draxis); nialamid (Niamid); pheneizine (Nardil) (Parke-Davis); pargyline (Euronyl) (Abbott); tranylcypromine (Parnate) (Smith-Kline Beecham); budipine (BY-701) (t-butyl-4,4 diphenyl piperidine) (Byk Gulden); caroxazone (Timostenil, Fl-6654) (Pharmacia Upjohn₇ Farmitalia); D-1711 (Biocodex); fezolamine (WIN-41528-2) ([N,N-dimethyl-3,4-diphenyl-1-H-pyrazole-1-propanamine-(E)-2-butenedioatel) (Sanofi); FLA-334 (RAN-113). (p-amino-amphetamine) (Astra); FLA-289 (FLA-299, FLA-365, FLA-384, FLA463, FLA-727) (p-rimethylamino-amphetamine) (Astra); K-11566 (Pharmacia Upjohn, Farmitalia); K-11829 (Pharmacia Upjohn, Farmitalia); metralindole; MPCPAM (Yissum); PharmaProjects 227 (Syntex/Roche); PharmaProjects 2806 (Fournier); PharmaProjects 1122; PharmaProjects 3311 (Roche); PharmaProjects 4433 (Roche); RS-2232 (Sankyo); UP-614-04 (Bristol-Myers), or hypricum perforatum.

It is understood that, although the terms, “inhibitor of monoamine oxidase” and “addictive substance, or derivative thereof” are singular, more than one inhibitor of monoamine oxidase and more than one addictive substances or derivative thereof, can be used concurrently in the methods of the invention.

The present invention is directed to the novel combination of a monoamine oxidase inhibitor administered together with an addictive substance, or non-addictive derivative thereof. This combination has striking therapeutic effects and, accordingly, is unexpectedly useful in treating addiction and related conditions. The clinical results disclosed herein demonstrate that administration of selegiline is indeed an effective therapy for obtaining a reduction in unwanted addictions and furthermore, that the combination of selegiline and nictoine administration is considerably more effective than administration of either two agents alone. Thus, the combination of a monoamine oxidase inhibitor with an addictive substance, or a non-addictive derivative thereof, is a highly effective mode of therapy to treat addiction. Although demonstrated herein for selegiline and nicotine, other combinations incorporating members of the same classes of compounds may be expected to be equally effective. Thus, the combination of selegiline and nicotine may be expected to reduce the level of addiction to other substances, including opiates, cocaine, amphetamines, or sweets. Alternatively, by way of example, the combination of selegiline with an amphetamine may be similarly effective in reducing the number of cigarettes smoked by an addicted smoker. It is well known that among the class of addictive substances, many compounds manifest cross-tolerance with one another, demonstrating that these compounds may behave as pharmacological analogues of one another. For treatment of any particular addictive disorder in any particular individual, the choice of which monoamine oxidase inhibitor combined with which addictive substance, or derivative thereof, will be determined by the nature of the disorder, the individual tolerance of the individual, and the knowledge and experience of the practitioner.

The inhibitor of monoamine oxidase inhibitor is administered in an effective amount, which is that amount necessary to alleviate, reduce, attenuate, or eliminate the symptoms associated with the addictive disorder, disorder or condition to be treated or to slow disease progression. The monoamine oxidase inhibitor can also be administered prophylactically, in order to prevent symptoms associated with the addicitve disorder or disease, or to delay onset of symptoms associated withdrawl or abstinence. If more than one inhibitor of monoamine oxidase inhibitor is used, the effective amount is that amount of the combination of inhibitor of monoamine oxidase inhibitors that is necessary to alleviate, reduce, eliminate, prevent, or delay onset of the symptoms associated with withdrawal or abstinence The effective amount will be determined on an individual basis, and will be based in part, on consideration of the particular monoamine oxdidase inhibitor, the individual's size and gender, the severity of the symptoms to be treated, the result sought, and the disease, disorder or condition to be treated or prevented. The effective amount will generally be an amount which decreases brain monoamine oxidase activity by at least about 10%, preferably at least about 30%, at least about 50%, at least 70%, and up to 100%. Usually, the effective amount will be between approximately 0.001 mg/kg to 1 g/kg, preferably about 0.01 mg/kg to 10 mg/kg. For example, it was found that if the inhibitor of monoamine oxidase inhibitor is clorgyline, the preferred effective amount in mice to inhibit 90% of striatal inhibitor of monoamine oxidase while maintaining 50% of intestinal inhibitor of monoamine oxidase activity is 0.6 mg/kg. One of ordinary skill in the art is able to extrapolate the preferred effective amount in a subject. The effective amount can be determined by one of ordinary skill in the art, employing such factors and using no more than routine experimentation.

The effective amount can be administered in a series of doses separated by appropriate intervals, such as hours, days, or weeks. Alternatively, the effective amount can be administered as a sustained release dose, such as by a controlled-release dosage formulation.

The monamine oxidase inhibitor, or inhibitors, is administered nasally, via a respiratory route or an intrapulmonary route, via an oral or enteral route, or via a parenteral route, i.e., by any means other than through the gastrointestinal tract or lungs. For example, the MAO inhibitor can be administered subcutaneously, transdermally, intradermally, intravenously, intramuscularly, intraperitoneally, topically, rectally, vaginally, or via an implanted reservoir. In a preferred embodiment, the MAO inhibitor is administered intradermally or transdermally. The term “transdermal” includes passive transdermal and also active electrotransdermal, e.g., iontophoretic or electroosmotic, delivery Representative intradermal, transdermal delivery and electrotransdermal and other transdermal delivery means are described in the art. A variety of compositions of inhibitors of monoamine oxidase, in particular selegiline, are disclosed in the art, including tablets, pills, capsules, powders, aerosols, suppositories, skin patches, parenterals, and oral liquids, including oil-aqueous suspensions, solutions, emulsions, and selegiline-containing sustained release (long acting) formulations and devices.

The compounds and methods of this invention can be adapted to the formulation and use of the therapeutic compositions comprising an inhibitor of monoamine oxidase and an addictive substance, or non-addictive derivative thereof, in admixture with conventional excipients, i.e. pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

Practice of the inventive method comprises administrating to a subject in need thereof of a first compound selected from the group of monoamine oxidase inhibitors in combination with a second compound selected from the group of addictive substances, including but not only, nicotine, opiates, amphetamines, and cocaine. The first component may be either a specific or non-specific inhibitor of monoamine oxidase isozymes. When administered in different vehicles through either the same or different routes of administration the method is known herein as co-administration; both administration of the compounds via the same vehicle or co-administration through different vehicles are acceptable forms of administration when implementing the invention.

For the purpose of implementing the invention, any compound known as a inhibitor of monoamine oxidase and any compound or composition known to be an addictive substance, or derivative thereof, are acceptable.

While the examples provided herein describe use of the monoamine oxidase inhibitor in oral administration and the addictive substance in transdermal administration, the success described affords good evidence to suppose that other routes of administration, or combinations with other pharmaceutical preparations, would be at least equally successful. The route of administration, e.g., topical, transdermal, parenteral, oral, or inhalant, and the dosage regimen will be determined by skilled clinicians, based on factors such as exact nature of the condition being treated, the severity of the condition, the age and general physical condition of the patient, and so on.

The inhibitor of monoamine oxidase inhibitor can be administered in dosage formulations containing conventional, non-toxic, physiologically-acceptable carriers, adjuvants, and/or vehicles. The formulation in which the inhibitor of monoamine oxidase inhibitor is administered will depend at least in part on the route by which it is administered. In implementation, more than one inhibitor of monoamine oxidase may be administered, including two or more monoamine oxidase inhibitors each of which exerting a selective effect on inhibition of one of the isozyme forms of the enzyme, i.e. monoamine oxidase A and monoamine oxidase B.

The daily dose can be administered in a single or multiple dosage regimen. Although the oral route of administration will generally be most convenient, drug may be administered by the parenteral, topical, transdermal dosage forms, including ointments, creams and patches, intraocular, buccal, sublingual, intranasal, inhalation, vaginal, rectal or other routes as well.

Oral dosage forms will most typically be used and are preferred but other dosage forms may also be employed and may permit, for example, a continuous release of relatively small amounts of the active ingredient from a single dosage unit, such as a transdermal patch, over the course of one or more days.

As used herein, the term “pharmaceutically effective amount” refers to an amount of a first compound, selected from the group on inhibitors of monoamine oxidase, and an amount of a second compound, selected from the group of addictive substances, or non-addictive derivatives thereof, which will produce the desired alleviation in symptoms or signs of substance addiction in a subject when administered as either a single composition, i.e. in the same vehicle, or when co-administered via either separate routes of administration or via separate vehicles. The doses utilized for any of the above-described purposes will generally be from 0.001 to about 1.00 milligrams per kilogram of body weight (mg/kg), administered up to 800 times per day for respiratory delivery, one to twenty times per day for comestible articles, one to six times per day for oral tablets or capsules, one to six times per day for injections, one to six times per day for transdermal patches or topical creams, one to six times per day for suppositories, or by continuous circulatory system infusion for pump delivery from an internal or external resevoir. When the compounds are present in the same vehicle, the ratio of monoamine oxidase inhibitor to addictive substance, or non-addictive derivative thereof, may range from 1:100 w/w to 100:1 w/w. Thus, for example, a transdermal patch for use in implementing the invention may comprise, for a 70 kg subject, from 0.7 to 70 mg selegiline and from 0.7 to 70 mg nicotine per patch dosage unit, applied from one to four times per day. Alternatively, by way of example, an oral dose of selegiline ranging from 0.7 to 70 mg may be co-administered to the subject together with a transdermal patch comprising from 0.7 to 70 mg of nicotine per patch dose unit, each of which dose may be administered at a rate of 1 to 6 times per day, in accordance with the needs and preferences of the subject or his/her practitioner.

As used herein, the term “pharmaceutically acceptable carrier” refers to any formulation which is safe, and provides the appropriate delivery for the desired route of administration of an effective amount of at least one compound of the present invention. As such, all of the above-described formulations of the present invention are hereby referred to as “pharmaceutically acceptable carriers.” This term refers to as well the use of buffered formulations wherein the pH is maintained at a particular desired value, ranging from pH 3.0 to pH 10.0, in accordance with the stability of the compounds and route of administration.

For parenteral application, particularly suitable are injectable, sterile solutions, preferably oily or aqueous solutions, as well as suspensions, emulsions, or implants, including suppositories. Ampoules are convenient unit dosages.

For application by inhalation, for delivery to the respiratory epithileum, in particular the lower respiratory tract and lungs, solutions or suspensions of the compounds mixed and aerosolized or nebulized in the presence of the appropriate carrier are suitable. Thus the carrier may be a vapor, a nebulized vapor, a. vapor containing particulate material, or a smoke.

For topical application, admixture of the compounds with conventional creams or delayed release patches is acceptable.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

Pharmaceutical compositions containing an inhibitor of monoamine oxidase and an addictive substance or non-addictive derivative thereof, can be prepared according to conventional techniques. For example, preparations for parenteral routes of administration of the combination, e.g., intramuscular, intradermal, subcutaneous, intravenous and intraarterial routes, can employ sterile isotonic saline solutions. Sterile isotonic buffered solutions can also be employed for intraocular administration.

Transdermal dosage unit forms of inhibitors of monoamine oxidase inhibitors can be prepared utilizing a variety of previously techniques known in the art. For example, a monolithic patch structure can be utilized in which the compound is directly incorporated into the adhesive and this mixture is cast onto a backing sheet. Alternatively, the drug can be incorporated as an acid addition salt into a multilayer patch which effects a conversion of the salt to the free base.

In a particularly preferred embodiment, the present invention is directed to a transdermal delivery composition comprising selegine and nicotine in a single patch for use in treating a condition in a human related to addiction to a substance and the desire to diminish or otherwise modify the intake of that substance. Transdermal patches comprising either nicotine or selegiline have been developed and one skilled in the art may readily adapt these carrier forms to a single patch comprising both compounds. Patches comprising a combination of selegiline, or another inhibitor of monoamine oxidase, and an addictive substance, or derivatives thereof, other than nicotine, are also objects of the invention, and may be readily prepared by one skilled in the pharmaceutical arts.

Several different pharmaceutical preparations of nicotine are available, including transdermal patches comprising nicotine, comestibles (gum for chewing) comprising nicotine, and inhalants for mucosal and pulmonary delivery comprising nicotine. These preparations are available for the purpose of treatment of addiction to tobacco products as nicotine replacement therapy.

In addition, nicotine may be administered by tablet form, for absorption through the gastrointestinal tract, although this route has generally not been preferred due to side effects and inferior absorption. However, for purposes of the present invention, this route of administration may also be considered a useful method of therapy when co-administered with a monoamine oxidase inhibitor.

Nicotine and at least one monoamine oxidase inhibitor, such as selegiline, may be co-administered though different routes of administration, as described herein for orally administered selegiline and transdermally administered nicotine, or, as an alternative, these compounds may be administered through the same route of administration for achieving the object of the invention which is to aid and facilitate for a subject the process of withdrawal from an addictive substance, in particular nicotine containing tobacco products. Thus, both selegiline and nicotine may be co-administered to a subject via transdermal route employing the use for this purpose of transdermal patches containing selegiline and nicotine to achieve the same results as described herein for co-administration of these compounds in the treatment of a subject in need thereof.

The present invention also provides for pharmaceutical compositions comprising both nicotine and at least one monoamine oxidase inhibitor. Thus, for purposes of implementing the invention, a pharmaceutical composition comprising nicotine and at least one monoamine oxidase inhibitor, such as selegiline, may be administered to a subject addicted to a substance, such as nicotine, or a nicotine-containing tobacco product, such as cigarettes, for the purpose of facilitating a desired reduction in the level of nicotine or tobacco product consumption.

A single pharmaceutical composition comprising nicotine, or a pharmacologically active derivative thereof, and a monoamine oxidase inhibitor, has distinct advantages to that of administering these therapeutic agents through different routes or through the same route of administration but as separate compositions. A pharmaceutical composition comprising both types of agents has the advantage of simplification of the treatment regimen with respect to the subject, who would therefore require the administration of a single tablet or patch comprising both nicotine and an effective monoamine oxidase inhibitor, such as selegiline, instead of two separate tablets or separate patches, for example. Combination of nicotine, or an pharmacologically equivalent derivative thereof, and an effective monoamine oxidase inhibitor as a single pharmaceutical composition affords an improved means for implementing the treatment of a subject in need of administration of both types of compounds for the purpose of treating addiction, since it is well known that administration of a single pharmaceutical composition is often better tolerated, enhances subject compliance, and prevents subject confusion when compared to the co-administration of two different compositions.

Successful use of the compositions and methods above requires employment of an effective amount of both the inhibitor of monoamine oxidase and the addictive substance or non-addictive derivative therof. Both the monoamine oxdiase inhibitor and the addictive substance may be equipotent in achieving the desired effect, yet the combination does not necessarily require a commensurately increased dosage to obtain the same level of therapeutic response confered by either compound individually. Thus, dosages necessary to attain a selegiline-like therapeutic effect may actually be lower when the monoamine oxidase inhibitor is employed in combination with nicotine. The same appears to be true for nicotine, as the combination with selegiline appears to allow for a dose reduction of nicotine sooner than when nicotine is used alone. Thus the risk of adverse effects of either the monoamine oxidase inhibitor or of the addictive substance are minimized due to the potency of the two when administered in combination.

Co-administration of pharmaceutically active compounds, i.e. drugs, is defined herein as administration to a subject of two or more active compounds wherein each compound is administered to a subject within a time proximity such that the subject is exposed to the effects of the drugs at the same time. Thus co-administration may be implemented by administering pharmaceutical compositions of two different drugs either simultaneously, or each drug preparation at different hours of the same day or week, provided that the subject is exposed to the effects of each compound at the same time. The effects of a pharmaceutically active compound on a subject may be measured either by subjective report, i.e. the subject reports a decreased craving for nicotine, or through measurement of tissue levels of the administered substance or metabolites thereof or through other means, such as measuring the level of inhibition of the activity of the enzyme monoamine oxidase or measuring the degree of occupancy of binding sites in the brain through the use of isotopically labeled monoamine oxidase compounds or derivatives thereof.

In preferred embodiment, the invention entails at least once daily administration of a monoamine oxidase inhibitors although alternate-dosing regimens may produce the same results in terms of subjective effects or measurable tissue levels, The rate of dosing of the monoamine oxidase inhibitor is also a function of the specific active compound employed. Thus, by way of example, meclobomide is typically administered twice a day while selegiline or pheneizine are typically administered once a day.

In broader use of the inventive composition, persons attempting to abstain from nicotine intake will probably self-medicate. A recommended dose for self-medication is likely a maximum of about six capsules per day, each comprising an inhibitor of monoamine oxidase, an addicitive substance or a derivative thereof, and a physiologically acceptable diluent or carrier, administered at the rate of about one capsule per hour to one per three hours. Alternatively, as one embodiment of the invention, a subject may partake of a nicotine-containing product such as a dispenser of nicotine in vapor, smoke, aerosol, particulate aerosol, or nebulized form, in a desired manner in accordance with the subjects's needs for self-medication while, at the same time, taking from one to six daily doses of a monoamine oxidase inhibitor.

The amount of monoamine oxidase inhibitor to be administered for treating a subject addicted to a substance or product, such as a tobacco product, is a function of several factors and may be modified in accordance with one or more of these factors to achieve the desired result. Among the factors which determine the amount of monoamine oxidase inhibitor to be administered are the specific type of monoamine oxidase inhibitor to administered, the traditional clinical or manufacturer recommended dose, the individual tolerance of the patient, the co-administration of other medications, and the extent of therapeutic effect achieved at any particular dose. Thus, a typical starting dose may be decreased due to patient intolerance, such as experiencing a side effect of the medication, or a typical starting dose may be increase due to lack of desired response.

The therapeutic response due to treatment by co-administration of a monoamine oxidase inhibitor with nicotine, according to the present invention, may be either a subjective report or objective measure of the level of consumption of the addictive substance. Thus, when used for treating a subject addicted to tobacco product, the response may be assessed by the subject's report of a decrease in the consumption of the tobacco product or a reduced sense of craving for the tobacco product, or both.

Another type of therapeutic response provided by implementation of the invention, which may be in addition to a reduction in craving, is the subject's experience of a reduction in other discomforting withdrawal signs or symptoms experienced by the subject who is in the process of attempting to discontinue, attenuate, or curtail his or her level of addictive substance consumption. Such a withdrawal state may be characterized by anxiety, irritability, poor concentration, restlessness, impatience, depressed mood, insomnia or hypersomnia, enhanced thirst, enhanced appetite, gain of body weight, sexual dysfunction, or craving or consumption of another substance or product, in particular a substance or product known to have addictive potential for the subject. It is known that, in subjects addicted to substances such as ethanol, cocaine, amphetamines, or opiates, craving for these addictive substances may be partially released by self-administration of a nicotine-containing tobacco product, in particular cigarette smoking

A significant advantage of the present invention is the co-administration of a monoamine oxidase inhibitor, such as selegiline, and nicotine replacement therapy, such as a transdermal nicotine patch, for treating a subject addicted to tobacco products and in need of specific therapy to reduce the level of consumption thereof, including achieving a state of complete abstinence. Moreover, while both monoamine oxidase inhibitors, as demonstrated herein for selegiline, and nicotine replacement therapies, such as administration of a transdermal patch, are effective modalities for a treating a subject addicted to tobacco products, the administration to a subject of the two types of drug together, i.e. co-administration of selegiline and nicotine, affords therapeutic results which surpass the efficacy of either form of therapy when administered alone. Thus the co-administration of a monoamine oxidase inhibitor and a nicotine replacement regiment, as disclosed below for co-administration to subjects in need thereof of selegiline and nicotine transdermal patch, comprises a synergistic combination previously unanticipated in the art.

Tablets, capsules, and comestible articles of the invention optionally include one or more pharmacologically suitable component or components such as buffering agent, preservative (e.g. antioxidants), excipients and/or tabletting agents, flavoring agents, and the like. Suitable such components are well-known to the tabletting art.

Some examples of suitable carriers, excipients, and diluents include lactose, dextrose, sucrose, sorbitol, mannitol, starches, gum, acacia, calcium phosphate, alginates, tragacanth, gelatin, calcium silicate, micro-crystalline cellulose, polyvinylpyrrolidone, cellulose, water syrup, methyl cellulose, methyl and propyl hydroxy-benzoates, talc, magnesium stearate, and mineral oil. The formulations can additionally include lubricating agents, wetting agents, emulsifying and suspending agents, preserving agents, sweetening agents, or flavoring agents. Compositions may be formulated so as to provide quick, sustained, or delayed release of the active ingredient after administration to the patient by employing procedures well known in the art.

The following formulation example is illustrative only and is not intended to limit the scope of the invention in any way.

### CLINICAL EXAMPLES

To demonstrate the therapeutic effect of selegiline and of a combination of selegiline and nicotine in the treatment of a subject addicted to tobacco products, the results of two experimental clinical trials are disclosed herein for the first time.

Experiment One

An experimental clinical trial was performed to assess the effect of selegiline alone and in combination with co-administration of nicotine. Neither the use of selegiline alone nor in combination with nicotine has been previously tested or implemented for the treatment of substance addiction and is disclosed herein for the first time.

In this prospective open trial subjects addicted to chronic cigarette smoking were administered either oral selegiline tablets (DEXXON, ISRAEL) 5 mg. b.i.d. or administered, for subjects 1 and 8, combined co-administration of both oral selegiline and nicotine replacement therapy via transdermal patch. Subjects were then monitored for their level of tobacco product consumption during the course of the next 8 weeks. The study protocol relied on the subjective report of the patient and measurement of urine cotinine levels if patient did not receive Nicotine Replacing Therpy (NRT). Reporting the level of cigarette consumption was elicited from the participating subjects at weekly intervals throughout the course of the study. Initial, trial entry levels of craving were determined by administration of the Fagerstrom Tolerance Score exam, while initial level of mood was monitored by administering the Beck Depression Inventory. These initial characteristics of the patient characteristics are summarized in Table 1.

TABLE 1
Initial Characteristics of the Trial Subjects

| | | | Average | | | Beck |
|---|---|---|---|---|---|---|
| Patient | | | Consumption | Pack | Tolerance | Depression |
| No. | Age | Sex | (cigarettes/day) | Years | Score^{†} | Inventory^{‡} |
| 1 | 40 | M | 20 | 20 | 7 | 2 |
| 2 | 39 | F | 25 | 25 | 6 | 7 |
| 3 | 30 | M | 15 | 10 | 4 | 9 |
| 4 | 55 | F | 20 | 40 | 6 | 2 |
| 5 | 39 | M | 20 | 22 | 5 | 20 |
| 6 | 55 | F | 30 | 50 | 7 | 0 |
| 7 | 37 | M | 20 | 20 | 8 | 6 |
| 8 | 35 | M | 20 | 16 | 5 | 0 |
| 9 | 39 | M | 25 | 30 | 7 | 3 |

^{†}Fagerström Tolerance Score (0-11); ^{‡}Beck Depression Inventory (0-63)

Following one week on the medication, the subjects were instructed to quit smoking on “quit day”, as predetermined at the initiation of the study. As shown in Table 2 below, only one subject taking selegiline alone out of six patients receiving selegiline successfully abstained from cigarette smoking (14% success). In comparisin, all patients (patients 1 and 8) taking both nicotine and selegiline successfully abstained from cigarette smoking (100% success). Three other patients did not respond and eventually left the study Patient No. 8 initially receiving both selegiline and nicotine replacement, had not smoked a single cigarette during the first eight weeks of the study. Patient No. 9 receiving just nicotine patch alone resumed smoking after the fifth week of abstinence, but following the addition of oral selegiline, reduced the daily consumption of cigarettes smoked. In addition, subject No. 4 reduced her daily cigarette intake even further upon adding administration of nicotine patch at the sixth week of the quit day. Later she discontinued the patch, expressing that she wished to continue smoking her three post-meal cigarettes per day.

Surprisingly, while the efficacy of tobacco replacement therapy, when administered as sole therapy in the form of transdermal patch, is known to be between 9-20%, and that for selegiline as sole therapy, as demonstrated above, is about 14%, as disclosed herein in Table 2, 100% of the subjects receiving the combined therapy from the beginning were treated successfully, i.e. abstaining or significantly reducing their level of cigarette smoking while undergoing co-administration of the two pharmaceutical compositions.

The results of the study presented herein demonstrate that adminstration of selegiline is effective treatment for substance addiction, namely cigarette smoking. Moreover, the combination of selegiline and nicotine is more superior to either drug alone when used for this purpose, and addition of selegiline to nicotine replacement therapy has a synergistic effect, particularly effective when used to treat smokers who wish to further attenuate their level of cigarette consumption when undergoing nicotine replacement therpay alone as an abstinence promoting therapy.

In this study, at follow-up one year from the initiation of treatment, subjects no. 1, 2, and 8 continued to demonstrate continuous abstinence from smoking, providing a success rate of 100% for the inventive combination of selegeline with nicotine patch compared to 14.2% for selegeline alone.

According to these results, the success rate of combination therapy is 100%, a value which is much higher than that reported for either selegeline alone (14.2%, see above) or nicotine patch alone (9.8%—N. Eng J Med 1999: 340:685-91), and which indicates a synergism for the inventive combination of selegeline and nicotine patch regimen.

TABLE 2
Selegiline Alone and In Combination with Nicotine

| Subject | | Weeks from Quit Day | | | | | | | | | | | |
|---|---|---|---|---|---|---|---|---|---|---|---|---|---|
| No. | | −2 | 0 | 1 | 2 | 3 | 4 | 5 | 6 | 7 | 8 | 9 | 1 year |
| 1 | Cig/wk | 140 | | 0 | 0 | 3 | 0 | 0 | 0 | 0 | — | | 0 |
| | med | - - | | SN | SN | SN | SN | SN | SN | SN | — | | |
| 2 | Cig/wk | 150 | | 0 | 0 | 0 | 0 | 0 | 0 | 0 | 0 | | 0 |
| | med | - - | | S | S | S | S | -- | -- | -- | -- | | |
| 3 | Cig/wk | 105 | | 0 | 0 | 0 | 0 | 0 | 0 | 0 | 0 | 0 | Sm |
| | med | - - | | S | S | S | S | S | S | S | S | | |
| 4 | Cig/wk | 140 - | | 21 | 21 | 25 | 25 | 25 | 14 | 21 | 21 | 21 | Sm |
| | med | - | | S | S | S | S | S | SN | S | S | S | |
| 5 | Cig/wk | 140 | | 140 | — | — | — | — | — | — | — | | Sm |
| | med | - - | | S | — | — | — | — | — | — | — | | |
| 6 | Cig/wk | 210 | | 210 | — | — | — | — | — | — | — | | Sm |
| | med | - - | | S | — | — | — | — | — | — | — | | |
| 7 | Cig/wk | 140 | | 35 | 70 | — | — | — | — | — | — | | Sm |
| | med | - - | | S | S | — | — | — | — | — | — | | |
| 8 | Cig/wk | 140 | | 0 | 0 | 0 | 0 | 0 | 0 | 0 | 0 | | 0 |
| | med | - - | | SN | SN | SN | SN | SN | SN | SN | SN | | |
| 9 | Cig/wk | 140 | | 0 | 0 | 0 | 0 | 0 | 21 | 35 | 60 | 25 | Sm |
| | med | - - | | N | N | N | N | N | N | N | N | SN | |

Legend
S = selegiline; N = nicotine; SN = selegiline and nicotine; -- = none
Med = medication; Cig/wk = cigarettes consumed per week; — = trial dropout
Sm = returned to smoke.

Experiment Two.

The second experiment is a randomized double-blind placebo controlled clinical trial wherein subjects received either selegiline in oral tablet form, or a placebo, initiating administration of this drug one week prior to the date set for self-induced reduction or cessation of tobacco product consumption, in this case cigarette smoking.

Subjects were admitted to the study after undergoing medical examination including anamnesis for other medical conditions and other forms of substance abuse and all signed informed consent, including the use of placebo in place of selegiline. All of the subjects admitted to the study were chronic cigarette smokers, consuming at least 15 cigarettes daily for the preceding year, but in otherwise general good physical and free of concomitant medication use. The study was approved and subjected to regulation by both a hospital Helsinki committee and the Israel Ministry of Health.

Subjects were assigned to one of two groups: oral selegeline tablet 5 mg (DEXXON, ISRAEL) twice a day for 26 weeks in combination with nicotine skin patch for 8 weeks or oral placebo twice a day for 26 weeks combined with nicotine skin patch for 8 weeks. Self-reported abstinence, urine cotinine levels, safety laboratory parameters and adverse events were monitored at regular intervals.

During the initial phase of the trial, half of the patients were administered either selegiline tablets taken as 5 mg tablet twice a day, once in the morning and once in the afternoon. Alternately, to the second half of patients, instead of selegiline, a placebo tablet was provided, identical in shape, colour, marking, weight, and size to the selegiline-containing tablets. In all cases the physician investigator supervising the trial was blinded to the identity of the active selegiline tablet vs the placebo tablet.

According to the initial results of this long term abstinence study, a high degree of success was noted for smokers treated with the combination therapy of oral selegeline and nicotine patch (first 8 weeks only) regimen.

From among a group of 45 smokers who participated in the long-term clinical trial, 14 smokers demonstrated continous abstinence from smoking after one year, as shown in Table 3 below (31% success). In this study, participants were interviewed once every two weeks for the first three months and then at six months and twelve months (where applicable) from the intiation of treatment. In addition to self-reporting, urinalysis for cotinine was negative on each of the four occasions conducted following conclusion of the initial eight week patch therapy.

According to these results, the success rate of combination therapy is 31% of Continous Abstinance, a value which is much higher than that reported for either selegeline alone (14.2%, see above) or nicotine patch alone (9.8%—reported in N. Eng J Med 1999: 340:685-91 in which identical protocol was used ). The combination of nicotine and selegeline exhibited a synergistic increase of 7% in the percent of patents with Continous Abstinance of one year. These results clearly indicate a synergism for the inventive combination of selegeline and nicotine patch regimen.

TABLE 3
Characteristics of Quitters (Continuous Abstinence from Smoking)
Treated by Oral Selegeline and Nicotine Patch Combination Therapy

| | | | | Continuous |
|---|---|---|---|---|
| Participant | | | | abstinence |
| no. | Sex | Age | Pack-years | (months) |
| 1 | F | 42 | 36 | 12 |
| 2 | M | 29 | 15 | 12 |
| 3 | F | 40 | 25 | 12 |
| 4 | F | 38 | 30 | 12 |
| 5 | M | 35 | 24 | 12 |
| 6 | F | 42 | 13 | 12 |
| 7 | M | 47 | 29 | 12 |
| 8 | M | 51 | 40 | 12 |
| 9 | M | 36 | 20 | 12 |
| 10 | M | 47 | 45 | 12 |
| 11 | M | 38 | 29 | 12 |
| 12 | F | 43 | 48 | 12 |
| 13 | M | 43 | 26 | 12 |
| 14 | F | 49 | 90 | 12 |

What we claim is:

1. A composition for the treatment of addiction to an addictive substance comprising an effective amount of an inhibitor of monoamine oxidase, an effective amount of an addictive substance or derivative thereof, and a suitable diluent or carrier.

2. A composition according to claim 1, wherein said inhibitor of monoamine oxidase is selegiline and said addictive substance, or derivative thereof, is nicotine.

3. A composition according to claim 1, wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

4. A composition according to claim 3, wherein said composition is administered in an aerosol, a nebulized vapor, a smoke or a transdermal patch.

5. A composition according to claim 1, wherein said carrier is suitable for oral administration.

6. A composition according to claim 5, wherein said composition is administered in a tablet, a capsule, or a comestible article.

7. A composition according to claim 2 wherein said selegiline is administered via a tablet or a capsule and said addictive substance is administered via a transdermal patch.

8. A method of treating a subject addicted to an addictive substance or product comprising the step of administering to the subject in need thereof a pharmaceutical composition comprising a therapeutically effective amount of an inhibitor of monoamine oxidase and a therapeutically effective amount of an addictive substance, thereby attenuating consumption of said substance or product.

9. A method according to claim 8 wherein said inhibitor of monoamine oxidase and said addictive substance, or derivative thereof, are administered via the same vehicle or co-administered via different vehicles.

10. A method according to claim 8 wherein said inhibitor of monoamine oxidase and said addictive substance, or derivative thereof are administered via the same route of administration or via different routes of administrations.

11. A method according to claim 10, wherein said pharmaceutical composition is administered via parenteral administration, including intradermal, subcutaneous, intramuscular, intravenous, intrathecal, sublingual, rectal, vaginal, intraocular, transdermal, respiratory mucosal, or pulmonary routes of administration.

12. A method according to claim 11, wherein said pharmaceutical composition is administered via an aerosol, a nebulized vapor, a smoke or a transdermal patch.

13. A method according to claim 10, wherein said pharmaceutical composition is administered via oral administration.

14. A method according to claim 13, wherein said pharmaceutical composition is administered via a tablet, a capsule, or a comestible article.

15. A method according to claim 8 wherein said inhibitor of monoamine oxidase is selegiline and said addictive substance is nicotine.

16. A method according to claim 15 wherein selegiline is administered via oral administration.

17. A method according to claim 15 wherein nicotine is administered via a transdermal patch.

18. A method of treating the symptoms of nicotine withdrawal comprising the step of administering to the subject in need thereof a pharmaceutical composition comprising a therapeutically effective amount of an inhibitor of monoamine oxidase and a therapeutically effective amount of nicotine, thereby alleviating the symptoms of nicotine withdrawal.

19. A method according to claim 18 wherein said inhibitor of monoamine oxidase and said nicotine, are administered via the same vehicle or co-administered via different vehicles.

20. A method according to claim 18 wherein said inhibitor of monoamine oxidase and said nicotine are administered via the same route of administration or via different routes of administrations.

21. A method according to claim 18, wherein said pharmaceutical composition is administered via parenteral administration, including intradermal, subcutaneous, intramuscular, intravenous, intrathecal, sublingual, rectal, vaginal, intraocular, transdermal, respiratory mucosal, or pulmonary routes of administration.

22. A method according to claim 21, wherein said pharmaceutical composition is administered via an aerosol, a nebulized vapor, a smoke or a transdermal patch.

23. A method according to claim 18, wherein said pharmaceutical composition is administered via oral administration.

24. A method according to claim 23, wherein said pharmaceutical composition is administered via a tablet, a capsule, or a comestible article.

25. A method according to claim 18 wherein said inhibitor of monoamine oxidase is selegiline.

26. A method of treating a subject addicted to a tobacco product comprising the step of administering to the subject in need thereof a pharmaceutical composition comprising a therapeutically effective amount of an inhibitor of monoamine oxidase, and a therapeutically effective amount of nicotine, thereby attenuating tobacco consumption.

27. A method according to claim 26 wherein said inhibitor of monoamine oxidase is selegiline.

28. A method according to claim 26 wherein said inhibitor of monoamine oxidase and said nicotine, are administered via the same vehicle or co-administered via different vehicles.

29. A method according to claim 26 wherein said inhibitor of monoamine oxidase and said nicotine are administered via the same route of administration or via different routes of administrations.

30. A method according to claim 26, wherein said pharmaceutical composition is administered via parenteral administration, including intradermal, subcutaneous, intramuscular, intravenous, intrathecal, sublingual, rectal, vaginal, intraocular, transdermal, respiratory mucosal, or pulmonary routes of administration.

31. A method according to claim 30, wherein said pharmaceutical composition is administered via an aerosol, a nebulized vapor, a smoke or a transdermal patch.

32. A method according to claim 26, wherein said pharmaceutical composition is administered via oral administration.

33. A method according to claim 32, wherein said pharmaceutical composition is administered via a tablet, a capsule, or a comestible article.
